(19) United States
(12) Patent Application Publication
(10) Pub. No.: US 2014/0303386 A1
(43) Pub. Date: Oct. 9, 2014

(54) COMPOSITIONS AND METHODS FOR ENHANCING LIPID PRODUCTION IN MICROALGAE VIA INDUCTION OF CELL CYCLE ARREST

(71) Applicants: Paul G. Falkowski, Princeton, NJ (US); Joomi Kim, Highland Park, NJ (US)

(72) Inventors: Paul G. Falkowski, Princeton, NJ (US); Joomi Kim, Highland Park, NJ (US)

(73) Assignee: RUTGERS, THE STATE UNIVERSITY OF NEW JERSEY, New Brunswick, NJ (US)

(21) Appl. No.: 14/240,979

(22) PCT Filed: Aug. 24, 2012
(86) PCT No.: PCT/US12/52215

§ 371 Date: Jun. 24, 2014

Related U.S. Application Data
(60) Provisional application No. 61/527,328, filed on Aug. 25, 2011.

(51) Int. Cl. C12P 7/64

(52) U.S. Cl. CPC C12P 7/64
USPC 554/1; 435/134

(57) ABSTRACT

Methods for enhancing lipid production in marine algae and compositions comprising lipids obtained therefrom are provided.

This application claims priority to U.S. Provisional Application No. 61/527,328 filed Aug. 25, 2011, the entire contents being incorporated herein by reference.

Pursuant to 35 U.S.C. §202(c), it is acknowledged that the U.S. Government has rights in the invention described, which was made with funds from the National Science Foundation, Grant No. DGE 0903675.

### FIELD OF THE INVENTION

This invention relates to the fields of lipid metabolism and biofuel production. More specifically, the invention provides algal cells and methods for treating and culturing the same which enhance endogenous lipid levels thereby facilitating production of biodiesel fuels.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

There has been tremendous interest in using microalgae as feedstocks for a renewable source of biofuel. Microalgae have the ability to accumulate up to 50% of their dry cell weight in lipids, primarily in the form of triacylglycerol (TAG) storage lipids (Hu et al. 2008). Under optimal growth conditions however, TAGs are only produced in small quantities; cells only accumulate large quantities of TAGs under stress conditions like nutrient limitation, temperature, pH, or light stress. Lipid accumulation under nutrient limitation is a well-studied phenomenon; a trend towards more accumulation of lipid is observed in microalgae in response to nitrogen deficiency (Shifrin and Chisholm 1981, Roessler 1990). In diatoms that require silicon, silicon deficiency leads to an accumulation of storage lipids in diatom cells (Roessler 1988). Phosphorus limitation also promotes lipid accumulation in some species (Hu et al. 2008).

Although nutrient limitation leads to increased synthesis of TAGs in microalgae, cells lose some photosynthetic capacity as a result of nutrient deficiency; for example under nitrogen deprivation, protein synthesis decreases, but in such a way that tends to impair photosynthetic proteins preferentially (Falkowski and Raven 1997). This leads to lipid yields being lower than theoretical maximums. Accordingly a need exists in the art for means to coerce microalgae into a lipid-accumulating state, without lowering photosynthetic capacity or output.

### SUMMARY OF THE INVENTION

In accordance with the present invention, a method for increasing lipid production in algae is provided. In this method, the G1 phase of the cell cycle of microalgae is elongated, either through genetic modification or chemical means, such that photosynthetic capacity is redirected away from cell division, and towards lipid synthesis.

This can be done by arresting the cells in the cell cycle, via inhibition of cyclin-dependent kinase (CDK) activity: either through chemical inhibition of CDKs, or by knockdown of CDKs or cyclin genes. CDKs are required for cell cycle progression, and cyclins are small subunit proteins that bind to CDKs to activate them, and therefore required for CDK activity. Suppression of either CDKs or cyclins leads to cell cycle arrest or interruption. An exemplary method comprises culturing microalgae in the presence of an effective amount of a cyclin dependent kinase (CDK) inhibitor, resulting in cell cycle arrest and elevated fatty acid accumulation, when compared to microalgal cells grown in the absence of said inhibitor, while not adversely affecting photosynthetic capacity of inhibitor treated cells. In another method, algae are genetically modified so that one or more of their CDKs or cyclins can be suppressed. In one embodiment of such a method (RNA-mediated interference, or RNAi), constructs expressing anti-sense or inverted repeat-containing RNAs, specific to the CDK or cyclin sequence to be inactivated, are introduced into the algal cell. In a preferred embodiment of the method, the construct is under the control of an inducible promoter, so that gene suppression can be induced at a desired time (after cell cultures are grown to an optimal density). Once cells are induced to have their CDK(s) or cyclin(s) silenced, they arrest in the cell cycle, and accumulate more fatty acids. The method may also comprise harvesting said fatty acids from said cells. In another embodiment of the invention, a CDK inhibitor is applied to algal cells, and said treated cells accumulate more fatty acids than untreated cells; in some cases 80% more. In another aspect of the method, the inhibitor is added to the culture during exponential growth phase. In a preferred embodiment of the invention, the harvested fatty acids are employed as compositions for biofuel production.

### BRIEF DESCRIPTION OF THE DRAWING

FIG. 1 is a graph showing fatty acid content per cell and specific growth rate vs. Nu2058 dose.

### DETAILED DESCRIPTION OF THE INVENTION

The eukaryotic cell cycle can be divided into four phases. In the S (synthesis) phase, the entire genome is duplicated. In this phase, cellular energy is used to synthesize enzymes required for nucleotide synthesis, followed by DNA replication, proof-reading the finished product, and correcting detected mistakes. During the M (mitotic) phase the parental cell utilizes energy to compact the entire duplicated genome into chromosomes, produce a complex protein scaffold, the mitotic spindle, to attach, align and pull apart the duplicated chromosomes. This is followed by cytokinesis, when the cell divides, forming two identical cells. The G1 (gap) phase is the period between M and S phases and comprises the longest phase of the cell cycle. Its length depends on the type of cell, as well as its environment. If sufficient nutrition or growth stimulus is not available, cells will stop in G1 and not enter S phase (A. Murray, T. Hunt, The Cell Cycle: An Introduction (Freeman Press, New York, ed. 1st, 1993)). Following exit from M, cells can escape from the cell cycle into a non-dividing state or resting state; this is usually followed by terminal differentiation in the case of mammalian cells. The G2 phase is the period after S phase and before mitosis. This phase is generally short, as cells tend to divide soon after S phase is complete. Each phase of the cell cycle is characterized by a unique pattern of CDK activity (C. J. Sherr, Cell 73, 1059-65 (1993); C. J. Sherr, Cell 79, 551-5 (1994); and S. van den Heuvel, E. Harlow, Science 262, 2050-4 (1993)).

Because DNA replication and cell division require significant energy, redirecting this energy into lipid synthesis, and away from cell cycle progression, is desirable for biodiesel applications. This invention provides a way to optimize lipid production by preventing microalgal cells from progressing through the cell cycle by arresting them in G1. G1 is a metabolically active state, but one where energy does not have to be “wasted” on DNA or cell division; elongation of G1 phase therefore optimizes lipid production. The results presented herein reveal that inhibition of cyclin-dependent kinases leads to G1 arrest and increased lipid accumulation in microalgae. G1-arrested cells continue to photosynthesize, but photosynthetic products are redirected into lipid synthesis at the expense of nitrate assimilation, protein synthesis, and cell division; that is, lipid synthesis becomes a significant “sink” for electrons in G1-arrested cells. This method does not lead to a decrease in the cell's photosynthetic capacity. A method to optimize lipid production in microalgae would therefore be to grow cells to some optimal level, then apply a CDK inhibitor or inhibit CDK activity by inducing suppression of a CDK or cyclin gene, so that they accumulate lipid over the course of a day or two, and then to harvest the cells. CDKs are serine-threonine kinases that regulate the cell cycle by activating (phosphorylating) other proteins involved in cell cycle progression. Their activities are dependent on cyclins, which bind to CDKs to activate them. A CDK bound to the correct cyclin forms an active CDK-cyclin complex. Cyclins are small proteins whose levels go up and down in a cell cycle dependent manner. Both CDKs and cyclins are required for CDK activity and cell cycle regulation, and suppression of one of them will lead to cell cycle interruption, and concomitant increased lipid accumulation. This discovery provides an efficient and reliable approach for optimizing lipid production in algae for the purpose of making stocks for biodiesel production.

One way to induce suppression of a CDK or cyclin gene is to introduce constructs expressing anti-sense or inverted repeat-containing RNAs, specific to the CDK or cyclin sequence to be inactivated, into the algal cell. This results in double-stranded RNA (dsRNA) precursors that will activate an endogenous silencing process in the cell, RNA-mediated interference (RNAi), in which the dsRNA are separated into single strands, integrated into a complex (RNA-induced silencing complex, or RISC), then base-paired with endogenous complementary mRNA (the mRNA of the gene to be inactivated). The result is double stranded RNA that gets recognized and cleaved by specific nucleases in the cell, and expression of the CDK or cyclin gene is prevented. If the CDK or cyclin suppressed is required for cell cycle progression from G1 to S phase, this will lead to G1 arrest in the cell, and concomitant increase in lipid accumulation in the cell.

The following definitions are provided to facilitate an understanding of the present invention.

“Cyclin-dependent kinases (CDKs)” are a family of serine-threonine kinases that trigger progression through the cell cycle.

“Cyclins” are small regulatory proteins (ranging in size from 35 to 90 kDa) that bind to CDKs to activate them. Their levels are controlled by transcription and degradation in a cell cycle dependent manner.

A “CDK inhibitor” is any molecule that is useful to inhibit CDKs in targeted microalgae. Such molecules block progression through the cell cycle, thereby enhancing lipid production. A CDK inhibitor can be a small molecule, a nucleotide or nucleoside analog, an inhibitory nucleic acid or a protein or peptide that is effective to interfere with CDK action in algae.

“CDK gene suppression” refers to any genetic modification that prevents the expression of a CDK gene. This includes methods to prevent transcription and/or translation of the CDK gene.

“Cyclin gene suppression” refers to any genetic modification that prevents expression of a cyclin gene. Preventing the expression of certain cyclins will prevent activation of CDKs, which will result in inhibition of CDK activity, and cell cycle arrest.

“G1 arrest” refers to a biochemical mechanism, responsive to diverse governing conditions (DNA damage, contact inhibition, growth factors, etc.) that control cellular progress through the G1 phase of the cell cycle. It describes a cell's state when it is prevented from progressing through the next phase of the cell cycle (S phase). G1 progress is controlled by the phosphorylation state of cyclin/CDK complexes.

The present invention also includes a biomass produced by one of the methods of the invention.

Cultivation conditions consistent with the organisms and methods of the present invention may be accomplished by methods described herein and methods known in the art and include the methods disclosed in U.S. Pat. No. 5,130,242, U.S. Pat. No. 5,407,957, U.S. Pat. No. 5,397,591; U.S. Pat. No. 5,492,938; and U.S. Pat. No. 5,711,983, and optimal conditions may readily be determined by those skilled in the art. Briefly, cultivation may be accomplished in any suitable fermentor, preferably in either a stirred tank fermentor or an air lift fermentor, which provide a source of oxygen to the microorganisms. The agitation of the microorganism should be maintained at a level such that while dissolved oxygen concentrations are sufficient to support the growth of the culture and production of the desired fatty acids, the agitation does not shear or otherwise damage the microorganisms. Preferred levels of dissolved oxygen are at least 10% of air saturation level. More preferably, levels of dissolved oxygen are maintained from about 10% to about 50% of air saturation levels.

Cultivation may be carried out at any life-sustaining temperature. Generally, microorganisms will grow at temperatures ranging from about 15° C. to about 34° C. Preferably the temperature is maintained at about 20° C. to about 28° C.

The organisms may be harvested by conventional means, known to those of skill in the art, such as centrifugation, flocculation, or filtration, and can be processed immediately or dried for future processing. In either event, lipid may be extracted. As used herein, the term “lipid” includes phospholipids; free fatty acids; esters of fatty acids; triacylglycerols; diacylglycerides; monoacylglycerides; lysophospholipids; soaps; phosphatides; sterols and sterol esters; carotenoids; xanthophylls (e.g., oxycarotenoids); hydrocarbons; and other lipids known to one of ordinary skill in the art. Different types or components of the lipids can be extracted, depending on the extraction technique that is used. Lipids can be extracted with an effective amount of solvent. Suitable solvents can be determined by those of skill in the art. Polar lipids (e.g., phospholipids) are generally extracted with polar solvents (e.g., chloroform/methanol) and neutral lipids (e.g., triacylglycerols) are generally extracted with nonpolar solvents (e.g., hexane). A preferred solvent is pure hexane. A suitable ratio of hexane to dry biomass is about 4 liters of hexane per kilogram of dry biomass. The hexane preferably is mixed with the biomass in a stirred reaction vessel at a temperature of about 50° C. for about 2 hours. After mixing, the biomass is filtered and separated from the hexane containing the oil. The hexane is removed from the oil by distillation techniques known to those of skill in the art. Conventional oilseed processing equipment is suitable to perform the filtering, separation and distillation. Additional processing steps, known to those of skill in the art, can be performed if required or desirable for a particular application. Alternative methods for lipid recovery are described in the following references which are incorporated by reference herein in their entirety: PCT Publication WO 0176715, entitled “Method for the Fractionation of Oil and Polar Lipid-Containing Native Raw Materials”; PCT Publication WO 0176385, entitled “Method For The Fractionation Of Oil And Polar Lipid-Containing Native Raw Materials Using Alcohol And Centrifugation”; PCT Publication WO 0153512, entitled “Solventless Extraction Process.”

The present invention, while disclosed in terms of specific organisms and methods, is intended to include all such methods and strains obtainable and useful according to the teachings disclosed herein, including all such substitutions, modifications, and optimizations as would be available expedients to those of ordinary skill in the art. The following examples and test results are provided for the purposes of illustration and are not intended to limit the scope of the invention.

### EXAMPLE I

### Cell Cycle Interruption Leads to Increased Lipid Synthesis in Microalgae

Application of a purine analog that inhibits cyclin-dependent kinases leads to cell cycle (G1) arrest in microalgae and increased lipid accumulation. G1-arrested cells continue to photosynthesize, but energy is redirected into lipid synthesis at the expense of nitrate assimilation, protein synthesis, and cell division. G1-arrested cells synthesize and accumulate 80% more fatty acids compared to cells growing normally.

### Experimental Conditions

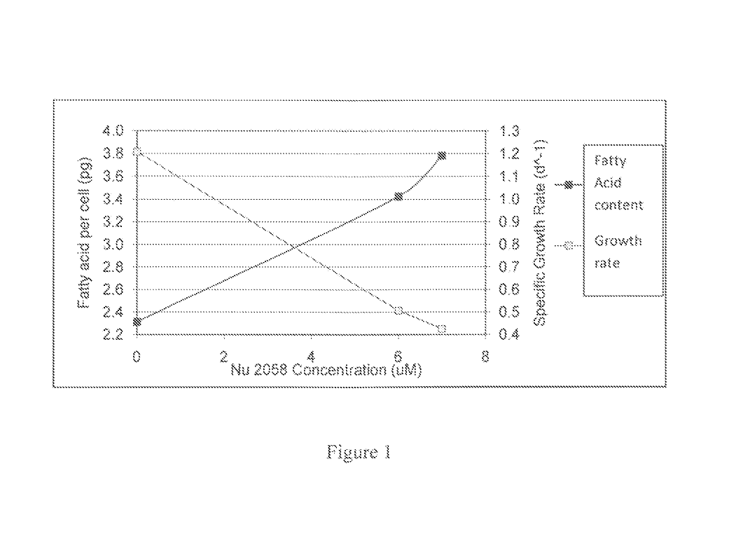

A purine analogue that binds to CDKs 1 and 2 in animals, NU 2058 (6-(Cyclohexylmethoxy)-9H-purin-2-amine), was used to cause cell cycle arrest in the marine diatom Phaeodactylum tricornutum. Cultures were grown in continuous light at approximately 70 μmol quanta m⁻²s⁻¹, in nutrient-replete conditions (F/2 media). All cultures (drugged and undrugged) had a final DMSO concentration of 1 μl/ml. The CDK inhibitor was applied when cells were growing exponentially (cell concentration was between 250,000-700,000 cells/ml). Application of the inhibitor led to a dose-dependent decrease in specific growth rate (FIG. 1). Cell cycle analysis was performed to confirm that cells were arrested in G1 phase; cells were fixed in methanol, washed in filtered PBS, treated with RNase A, and DNA was stained with propidium iodide before being analyzed by flow cytometry. The percentage of cells arrested in G1 phase determined total fatty acid per cell accumulated; a higher dose of inhibitor led to a higher percentage of cells arrested in G1, and concomitant higher fatty acid content per cell. A concentration of 7 μM led to an 80% increase in fatty acid synthesis over a period of one day, compared to untreated cells.

Protein synthesis in treated cells was about 18% lower than in untreated cells, and nitrate assimilation (measured as nitrate reductase activity) in treated cells was about 35% of the activity of untreated cells. Measures of photosynthetic health (Fv/Fm, chlorophyll per cell, a*) showed that there were no appreciable differences in photosynthetic capacity between treated and untreated cells.

While the CDK inhibitor NU58 is exemplified herein, the skilled person is aware that CDK inhibition can be achieved by genetic modification (suppression of a CDK or cyclin gene), through RNAi or other methods, and of many other molecules that are effective for inhibiting CDK action. These include, without limitation, those described in U.S. Pat. Nos. 7,511,049; 7,514,442; 5,672,508; 7,598,260 and 7,605,175. Other inhibitors include, without limitation, AT7519, PHA793887, roscovitine, purvalanol A, and purvalanol B.

As mentioned above, inhibition of CDK expression can be achieved via silencing with targeted nucleic acid based constructs. Given that sequence information for the nucleic acid encoding CDK(s), particularly CDK1 and CKDK2, from microalgae species are set forth in GenBank, the design of suitable siRNA molecules and/or antisense molecules useful for silencing or inhibiting expression of these particular gene is readily achievable without undue experimentation. Commercial vendors, such as Dharmacon®, are available to provide pools of siRNA based on known sequence information. For long term expression, cloning of such small molecules into suitable expression vectors is alwo well within the purview of the skilled artisan.

After a suitable period of culture, lipids can be harvested by solvent extraction and recycling as is well known to those skilled in this art area. See for example P. Prabakaran et al., Lett Appl Microbiol. 2011 August;53(2):150-4 Epub 2011 Jun. 13; Y. Gong et al. Biotechnol Lett. 2011 July;33(7):1269-84. Epub 2011 Mar. 5 and B. D. Wahlen et al., Bioresour Technol. 2011 February; 102(3):2724-30. Epub 2010 Nov. 12.

### REFERENCES

Shifrin, N. S. and Chisholm, S. W. (1981) Phytoplankton lipids: interspecific differences and effects of nitrate, silicate and light-dark cycles. J. Phycol. 17, 374-384.
Falkowski, P. G. and Raven, J. (1997) Aquatic Photosynthesis. Malden, M A: Blackwell Science.
Hu, Q., Sommerfeild, M., Jarvis, E., Ghirardi, M., Posewitz, M., Seibert, M., and Darzins, A. (2008) Microalgal triacylglycerols as feedstocks for biofuel production: perspectives and advances. The Plant Journal, 54, 621-639.
Roessler, P. G. (1988) Changes in the activities of various lipid and carbohydrate biosynthetic enzymes in the diatom Cyclotella cryptica in response to silicon deficiency. Arch. Biochem. Biophys. 267, 521-528.
Roessler, P. G. (1990) Environmental control of glycerolipid metabolism in microalgae: commercial implications and future research directions. J. Phycol. 26, 393-399.

While certain of the preferred embodiments of the present invention have been described and specifically exemplified above, it is not intended that the invention be limited to such embodiments. Various modifications may be made thereto without departing from the scope of the present invention, as set forth in the following claims.

1. A method for increasing lipid production in a marine alga culture comprising:
culturing microalgae in the presence of an effective amount of a cyclin dependent kinase (CDK) inhibitor, exposure to said inhibitor resulting in elevated fatty acid accumulation when compared to microalgal cells grown in the absence of said inhibitor while not adversely affecting photosynthetic capacity of inhibitor treated cells.

2. The method of claim 1, comprising harvesting said fatty acids from said cells.

3. The method of claim 1, wherein said inhibitor is NU2058 and said treated cells accumulate 80% more fatty acids than untreated cells.

4. The method of claim 1 wherein said inhibitor is added to said culture during exponential growth phase.

5. The method of claim 1, wherein said alga comprise a nucleic acid construct encoding nucleic acid molecules effective to down modulate CDK expression.

6. The method of claim 5, wherein said nucleic acid molecule is selected from the group consisting of antisense molecules which specifically hybridize with CDK encoding nucleic acids, one or more siRNA molecules effective to silence CDK gene expression, said molecules optionally being expression in an expression vector.

7. The method of claim 6, wherein said nucleic acid construct comprises an inducible promoter operably linked to said nucleic acid molecule encoding sequences effective to down modulate CDK expression.

8. The method of claim 1, wherein said fatty acids are used for biofuel production.

9. The method of claim 1 1 through 7, wherein the microalgae is concentrated employed as a food grade dietary supplement.

10. The method of claim 1, wherein the microalgae is concentrated and employed in aquaculture or as animal feed.

11. The method of claim 1, wherein said microalgae is P. tricornutum.

12. The method of claim 1, wherein said microalgae is D. terciolecta.

13. A biomass produced by the method of claim 11, wherein lipids are optionally harvested from said biomass

14. A biomass produced by the method of claim 12, wherein lipids are optionally harvested from said biomass.
